(19) United States
(12) Patent Application Publication
(10) Pub. No.: US 2017/0326077 A1
(43) Pub. Date: Nov. 16, 2017

(54) HOMOGENOUS COMPOSITION AND METHODS OF MAKING THE SAME

(71) Applicant: BIO-UP MIMETIC TECHNOLOGIES, INC., La Habra, CA (US)

(72) Inventors: Crispin G.S. Eley, Fullerton, CA (US); Donald F. Hodgson, Fullerton, CA (US)

(21) Appl. No.: 15/668,679

(22) Filed: Aug. 3, 2017

Related U.S. Application Data
(62) Division of application No. 13/135,057, filed on Jun. 23, 2011.
(60) Provisional application No. 61/357,959, filed on Jun. 23, 2010.

(51) Int. Cl. A61K 31/05; A61K 38/43; A23L 33/12; A23L 27/00; A23P 10/30; A23L 33/15; A61K 38/13; A61K 31/685; A61K 9/127; A23L 3/3454; A23D 7/01; A23L 27/12; A61K 47/44; A61K 9/00; A23J 7/00; A23D 7/00; A23L 33/00

(52) U.S. Cl. CPC A61K 31/05; A61K 47/44; A61K 38/43; A23L 33/12; A23L 27/72; A23P 10/30; A23L 33/15; A61K 38/13; A61K 31/685; A61K 9/127; A23L 3/3454; A23D 7/011; A23L 27/12; A61K 9/00; A23J 7/00; A23D 7/00; A23V 2002/00; A23L 33/00

(57) ABSTRACT

A hydrated lecithin carrier vesicle composition includes a lecithin-derived membrane-forming lipid vesicle in conditioned water for incorporation of an active ingredient to form a dispersed composition. A method of making the hydrated lecithin carrier vesicle includes using lecithin having not more than about 80% w/w phosphatidylcholine in the presence of conditioned water.

### CROSS-REFERENCE TO RELATED APPLICATION(S)

The present application claims priority to and the benefit of U.S. Provisional Application Ser. No. 61/357,959, filed on Jun. 23, 2010, the entire contents of which are incorporated herein by reference.

### TECHNICAL FIELD

This application is directed to lecithin carrier compositions and methods of making lecithin carrier compositions.

### TECHNICAL BACKGROUND

The dispersion and stabilization of active ingredients may be desirable in order to store and manipulate these desired compounds in aqueous environments. Commonly used methods for dispersion have included emulsions, in which droplets of the active ingredient are dispersed and stabilized by a surfactant, or by milling or shearing of the desired compound into nanoparticles and dispersing the nanoparticles into a surfactant. However, these surfactant emulsions are often not stable, and the surfactant may be toxic or have undesired properties such as poor taste and/or the dispersion has cloudy appearance. These properties render these emulsions inadequate for dispersion of an active ingredient for consumption.

Phospholipids and other membrane-forming lipids are widely used to encapsulate active ingredients for transport in aqueous environments. In particular, phospholipid bilayer vesicles are formed when dried phospholipids are hydrated in aqueous solution, thereby generating concentric multiple phospholipid bilayers separated by aqueous compartments, known as multilamellar vesicles (MLVs). Phospholipids may also be manipulated to form unilamellar vesicles (UVs). These unilamellar vesicles together with the multilamellar vesicles can be categorized into three types--small unilamellar vesicles (SUVs) having a mean diameter in the range from 20 to 100 nm; large unilamellar vesicles (LUVs) having a mean diameter in the range from 150 to 1,000 nm; and multilamellar vesicles (MLVs) having a mean diameter in the range from 150 to 5,000 nm.

The phospholipid phosphatidylcholine (PC) is the basic component of commercial phospholipid vesicles, commonly with the addition of defined amounts of charged lipids such as phosphatidylglycerol. Lecithin is a mixture obtained from animal or plant sources by hydration of solvent-extracted oils that comprises acetone-insoluble phosphatides, the majority of which are phosphatidylcholine (PC), phosphatidylethanolamine (PE), and phosphatidylinositol (PI), combined with various amounts of other substances such as triglycerides, fatty acids, and carbohydrates.

Lecithin is a commonly used source of the phosphatidylcholine that is used in the preparation of phospholipid bilayers (Keller, B.C, 2001, Trends in Food Science Technology, 12, 25-31). Because the dispersion capabilities and stability of phospholipid vesicles has been shown to be dependent on the amount of phosphatidylcholine in the vesicles, high PC-content lipid mixtures (i.e., greater than 80% PC) are used for forming phospholipid vesicle carriers. Although attempts have been made to use a lower PC-content to form dispersed liposomes for encapsulating a curcumin compound, those attempts did not result in dispersed vesicles encapsulating the curcumin. Takahashi et al. J. of Oleo Sci., 2007, 56, 35-42; (attempting to make liposomes having low-PC content) and Takahashi et al. J. of Agr. and Food Chem., 57, 9141-9146 (stating and proving that low-PC content liposomes did not work) are both incorporated by reference in their entirety.

In many protocols, the phosphatidylcholine is isolated from lecithin by de-oiling methods using acetone, followed by ethanol extraction, and a final liquid chromatography step to obtain high content phosphatidylcholine (i.e. greater than 80% PC of the total lipid content). To inhibit hydrolysis of the phospholipid vesicles, ionic buffers are used to control and maintain the pH of the vesicle environment (Vernooij, E., et al., Journal of Controlled Release, 2002, 79, 299-303). However, high PC-content lecithin is costly to make, and methodologies requiring certain organic solvents render the composition not desirable for human consumption. In this regard, there is a need for a phospholipid vesicle carrier that provides a stable dispersion of active ingredients using the fewest components and methods that do not require toxic solvents or expensive high-PC content lipid mixtures.

### SUMMARY

In some embodiments of the present invention, a composition including a stable homogenous dispersion includes a vesicle having a membrane and an aqueous phase, the vesicle including lecithin having a phosphatidylcholine content of about 80 w/w % or less, and an active ingredient incorporated in the membrane of the vesicle; and the stable homogenous dispersion also includes conditioned water. In some embodiments, the conditioned water has less than 100 ppm hard ions and/or a conductivity of less than 20 microSiemens per centimeter. In some embodiments, the vesicle has a volume-weighted mean diameter of about 120 nm or less. In other embodiments, the membrane of the vesicle is a phospholipid bilayer.

The stable homogenous dispersion may be a transparent dispersion. The stable homogenous dispersion may also include a stablizing agent, e.g., a polysorbate or a polyoxyethylene alkyl ether.

In some embodiments the stable homogenous table dispersion may also include alcohol, and the alcohol may be a short chain aliphatic alcohol, e.g., methanol, ethanol, isomers of propanol or isomers of butanol.

In some embodiments, the active ingredient is a lipophilic compound, and the lipophilic compound may be at least one from olfactants, natural essences, essential oils, coloring agents, vitamins, vitamin salts, pharmacologically active vitamin metabolites, pharmacologically active vitamin metabolite salts, phytochemicals, oil-soluble acids, oil-soluble alcohols, essential fatty acids, primrose oil, safflower oil, fish oil, lipids from marine organisms, cyclosporin A, propofol, fat-soluble protease inhibitors, antiretroviral compounds, antibiotics, carotenoids, steroidal hormones, flavonoids, proteins, enzymes, coenzymes, paints, inks, and agrochemicals.

In other embodiments, a composition includes a vesicle including lecithin having a phosphatidylcholine content of about 80 w/w % or less, and an active ingredient; and the composition also includes conditioned water. In some embodiments, the membrane of the vesicle is a phospholipid bilayer. The composition may also include a stablizing agent, e.g., a polysorbate or a polyoxyethylene alkyl ether. In some embodiments, the composition may also include alcohol.

In still other embodiments, a composition, including a homogenous mixture includes lecithin having a phosphatidylcholine content of about 80 w/w % or less, an active ingredient, and an alcohol. The homogenous mixture may also include conditioned water, which may be present in an amount of about 10% or less by weight relative to the lecithin. The homogenous mixture may also include an oil. The alcohol of the homogenous mixture may be present in an amount of about 50% or less by weight relative to the lecithin.

In still other embodiments, a method of producing a vesicle carrier composition includes hydrating lecithin having a phosphatidylcholine content of 80 w/w % or less in conditioned water to form a lecithin vesicle having a membrane and an aqueous phase; and incorporating an active ingredient into the membrane of the lecithin vesicle to form a membrane loaded lecithin vesicle.

This method of producing a vesicle carrier composition may further include, prior to incorporating the active ingredient, processing the lecithin vesicle to make the lecithin vesicle unilamellar, and the processing may include homogenization or high shear mixing. In some embodiments, the conditioned water may include alcohol.

This method may further include adding a stablizing agent to the lecithin prior to hydration or to the lecithin vesicle prior to or after incorporation of the active ingredient. This method may further include, after incorporation of the active ingredient into the membrane of the lecithin vesicle, processing the membrane loaded lecithin vesicle to reduce a size of the membrane loaded lecithin vesicle, and the processing may include homogenization or high shear mixing.

In some embodiments this method of producing a vesicle carrier composition further includes drying the composition to a solid form and/or incorporating the composition into a cream or paste.

In still other embodiments, a method of producing a vesicle carrier composition having a membrane and an aqueous phase, includes mixing lecithin having a phosphatidylcholine content of about 80 w/w % or less, an active ingredient, and an alcohol to form a homogenous liquid mixture; and hydrating the homogenous liquid mixture with conditioned water to form a lecithin vesicle in which the active ingredient is incorporated into the membrane of the lecithin vesicle.

This method of producing a vesicle carrier composition may also include heating during, mixing an oil with, and/or mixing conditioned water with the mixing of the lecithin having a phosphatidylcholine content of about 80 w/w % or less, an active ingredient, and an alcohol. In some embodiments, the conditioned water may be provided up to about 10% by weight relative to the lecithin. In some embodiments, the alcohol is provided in an amount of about 50% or less by weight relative to the lecithin. In some embodiments, this method also includes, after hydrating the homogenous liquid mixture, processing the lecithin vesicle to make the lecithin vesicle unilamellar, and the processing may include homogenization or high shear mixing.

In still other embodiments, a method of forming a homogenous composition, includes mixing lecithin having a phosphatidylcholine content of 80 w/w % or less with an active ingredient and an alcohol, and the alcohol may be provided in an amount of about 50% or less by weight relative to the lecithin. This method may further include mixing conditioned water with the mixing of the lecithin having a phosphatidylcholine content of 80 w/w % or less with an active ingredient and an alcohol, and the conditioned water may be provided up to about 10% by weight relative to the lecithin.

In still other embodiments, a method of producing a vesicle carrier composition, includes hydrating lecithin having a phosphatidylcholine content of more than about 80 w/w % in conditioned water to form a lecithin vesicle having a membrane and an aqueous phase; and incorporating an active ingredient into the membrane of the lecithin vesicle to form a membrane loaded lecithin vesicle. In some embodiments, the lecithin of this method may include phosphatidylglycerol.

In some embodiments, the active ingredient of this method is a pharmaceutically active ingredient selected from the group consisting of cyclosporin A, propofol, fat-soluble protease inhibitors, antiretroviral compounds, antibiotics, carotenoids, steroidal hormones, flavonoids, enzymes, and coenzymes.

In some embodiments, this method further includes, prior to incorporating the active ingredient, processing the lecithin vesicle to make the lecithin vesicle unilamellar, and the processing may include homogenization and high shear mixing.

In some embodiments, this method further includes adding a stabilizing agent to the lecithin prior to hydration or to the lecithin vesicle prior to or after incorporation of the active ingredient. This method may also include, after incorporation of the active ingredient into the membrane of the lecithin vesicle, processing the membrane loaded lecithin vesicle to reduce a size of the membrane loaded lecithin vesicle, and the processing may include homogenization and high shear mixing.

In still other embodiments, a method of producing a vesicle carrier composition having a membrane and an aqueous phase, includes mixing lecithin having a phosphatidylcholine content of more than 80 w/w %, an active ingredient, and an alcohol to form a homogenous liquid mixture; and hydrating the homogenous liquid mixture with conditioned water to form a lecithin vesicle in which the active ingredient is incorporated into the membrane of the lecithin vesicle. In some embodiments, the lecithin of this method may include phosphatidylglycerol.

In some embodiments, the active ingredient of this method is a pharmaceutically active ingredient selected from the group consisting of cyclosporin A, propofol, fat-soluble protease inhibitors, antiretroviral compounds, antibiotics, carotenoids, steroidal hormones, flavonoids, enzymes, and coenzymes.

In some embodiments, this method further includes heating during, mixing an oil with, and/or mixing conditioned water with the mixing of the lecithin having a phosphatidylcholine content of more than 80 w/w %, an active ingredient, and an alcohol. In some embodiments, the conditioned water is provided up to about 10% by weight relative to the lecithin. In some embodiments, the alcohol is provided up to about 50% by weight or less by weight relative to the lecithin.

In some embodiments, this method further includes, after hydrating the homogenous liquid mixture, processing the lecithin vesicle to make the lecithin vesicle unilamellar, and the processing may include homogenization or high shear mixing.

### BRIEF DESCRIPTION OF THE DRAWINGS

FIG. 1A is a photograph of compositions of fish oil dispersed in hydrated lecithin carrier vesicles in conditioned water and (left to right) 0, 10, 20, 30 40 or 50% ethanol, according to embodiments of the present invention.

FIG. 1B is a graph reporting the turbidity of the compositions of fish oil shown in FIG. 1A, according to embodiments of the present invention.

FIG. 1C is a graph showing the particle size distribution data acquired from the compositions of fish oil shown in FIG. 1A, according to embodiments of the present invention.

FIG. 2A is photograph of compositions of fish oil dispersed in hydrated lecithin carrier vesicles in conditioned water and (left to right) 0, 10, 20, 30 40 or 50% ethanol, and polysorbate, according to embodiments of the present invention.

FIG. 2B is a graph measuring the turbidity of the compositions of fish oil shown in FIG. 2A, according to embodiments of the present invention.

FIG. 2C is a graph showing the particle size distribution data acquired from the compositions of fish oil shown in FIG. 2A, according to embodiments of the present invention.

FIG. 3 is a photograph of compositions of fish oil dispersed in hydrated lecithin carrier vesicles in conditioned water and 30% ethanol with and without polysorbate, according to embodiments of the present invention.

FIG. 4A is a photograph of compositions of essential oils dispersed in hydrated lecithin carrier vesicles in conditioned water and polysorbate at t=0 and t=12 months, according to embodiments of the present invention.

FIG. 4B is a graph showing the particle size distribution data acquired from the composition of essential oils at t=0, as shown in FIG. 4A, according to an embodiment of the present invention.

FIG. 4C is a graph showing the particle size distribution data acquired from the composition of essential oils at t=12 months, as shown in FIG. 4A, according to an embodiment of the present invention.

FIG. 5A is a photograph of a composition of hill oil dispersed in hydrated lecithin carrier vesicles in conditioned water and a stabilizing agent, according to an embodiment of the present invention.

FIG. 5B is a graph showing the particle size distribution data acquired from the composition of krill oil as shown in FIG. 5A, according to an embodiment of the present invention.

FIG. 6A is a photograph of a composition of fish oil dispersed in hydrated lecithin carrier vesicles in conditioned water and 30% isopropyl alcohol, according to an embodiment of the present invention.

FIG. 6B is a graph showing the particle size distribution data acquired from the composition of fish oil in 30% isopropyl alcohol as shown in FIG. 6A, according to an embodiment of the present invention.

FIG. 7A is a photograph of a composition of fish oil dispersed in hydrated lecithin carrier vesicles in conditioned water and 5% isobutyl alcohol, according to an embodiment of the present invention.

FIG. 7B is a graph showing the particle size distribution data acquired from the composition of fish oil in 5% isobutyl alcohol as shown in FIG. 7A, according to an embodiment of the present invention.

FIG. 8A is a photograph of compositions of hydrated lecithin carrier vesicles having (1) an essential oil (carvacrol) and polysorbate; (2) an essential oil; and (3) lecithin alone, (no essential oil), according to embodiments of the present invention.

FIGS. 8B is a graph showing the particle size distribution data acquired from the composition (1) of FIG. 8A having an essential oil and polysorbate, according to an embodiment of the present invention.

FIG. 8C is a graph showing the particle size distribution data acquired from the composition (2) of FIG. 8A having an essential oil, according to an embodiment of the present invention.

FIG. 8D is a graph showing the particle size distribution data acquired from the composition (3) of FIG. 8A having lecithin alone, according to an embodiment of the present invention.

### DETAILED DESCRIPTION

In aspects of the present invention, a hydrated lecithin carrier vesicle (HLCV) composition includes lecithin having a phosphatidylcholine content of at most about 80 w/w % and conditioned water. The conditioned water hydrates the lecithin to form an HLCV. In some embodiments, the HLCV may have at least one active ingredient dispersed therein. However, in some embodiments, the HLCV composition includes alcohol which can help form HLCV dispersions of the active ingredient and ensure proper hydration in conditioned water. In other embodiments, the HLCV composition further includes one or more stabilizing agents. In some embodiments, the active ingredient is solubilized in lecithin with or without alcohol in a homogenous liquid mixture. Other aspects of the present invention are directed to methods of making the HLCV compositions and homogenous mixtures.

In other embodiments of the present invention, a hydrated lecithin carrier vesicle (HLCV) composition consists essentially of a vesicle having a membrane and an aqueous phase, and conditioned water. In these embodiments, the term “consists essentially of” refers to the general absence from the composition of lecithin particles and lecithin-based particles (or nanoparticles) that are not vesicles, within the meaning of that term as defined below. However, these embodiments include the same vesicles as the embodiments described above and below, and can be made by any of the methods described below. Also, these embodiments can further include any of the below described active ingredients and/or other components (e.g., stabilizing agents, alcohols and/or oils). Indeed, other than generally excluding the presence in the compositions of non-vesicle lecithin-based materials (e.g., non-vesicle lecithin particles and nanoparticles, and/or nanocrystals of active ingredient that are coated with non-vesicle lecithin), these embodiments may have the same composition as the embodiments described above and below (e.g., they may be made using the same materials and methods, and may include the same active ingredients, and other components, such as alcohols, stabilizing agents, oils, etc.).

The use of lecithin to disperse active ingredients, according to this disclosure, has utility for compounds to be ingested and is also of utility in many other areas including, without limitation, the fields of agriculture, horticulture, nutraceuticals, pharmaceuticals (for the diagnosis, treatment and palliation of disease), cosmetics and personal care products, fragrances and color agents, environmental remediation, inorganic and composite materials, paints and inks, catalysis, and such other fields where a low cost natural dispersing agent is desirable. For all embodiments of this invention, it is contemplated that other agents, including water-soluble substances, may optionally be added to the hydrated lecithin carrier vehicle dispersions to enhance their suitability for use in a given application, for example addition of a water-soluble anti-oxidant such as ascorbic acid to improve shelf-life of a nutrition product. Selection of the specific additives will be obvious to those skilled in the art.

Aspects of this invention provide for the use of bulk food or industrial grade lecithin to solubilize water-insoluble and partially water-insoluble substances in a manner that is cost-effective for broad use in consumer products, including food, beverage and nutritional supplements, and in other applications where effective commercialization is dependent on the cost of raw materials that precludes the use of high PC lecithin (i.e. lecithin having more than 80 w/w % phosphatidylcholine).

As used herein, lecithin is defined as a complex mixture obtained from animal and plant sources by hydration of solvent-extract oils, as defined in the Joint World Health Organization/United Nations Food Safety Agency Evaluation Committee for Food Additives (JECFA). (Food and Agriculture Organization of the United Nations, Food and Nutrition Paper 52, “Compendium of Food Additive Specifications” (FNP 52), Addendum 2 (1993)), which is incorporated by reference in its entirety. This complex mixture comprises acetone-insoluble phosphatides including predominantly phosphatidylcholine, phosphatidylethanolamine, and phosphatidylinositol, as well as smaller amounts of triglycerides, fatty acids, and carbohydrates.

As used herein, vesicle is defined as a composition having a membrane-forming lipid component and an aqueous phase. In some embodiments, the membrane-forming lipid component is a phospholipid bilayer membrane. Also, as used throughout this disclosure and claims, the term “vesicle” and “vehicle” are used interchangeably.

As used herein, active ingredient refers to any compound that is selected to be and is capable of being incorporated into the vesicle. For example, in some embodiments, the active ingredient can be lipophilic which includes many amphiphilic compounds, as discussed below.

Lipophilic compounds are more soluble in fats, oils, lipids and organic solvents such as ethanol, methanol, ethyl ether, acetone, chloroform and benzene than in water. Within their structure, lipophilic compounds may contain hydrophilic moieties, such as the hydroxyl group in sterols and the carboxylic acid group in long chain fatty acids. In some embodiments, lipophilic compounds are incorporated with the membrane-forming lipid component of the vesicle. In some embodiments, lipophilic compounds have log P values in a range from about 0 to about 8, where the higher log P value corresponds to increased lipophilicity. In some other embodiments, the lipophilic active ingredient has a log P value range from about 2 to about 7.

As used herein, the term “lipophilic compounds” and “lipophilic active ingredient” are used interchangeably, and refer to compounds having greater solubility in organic solvents, fats and oils, than in water. The term “lipophilic” also encompasses many amphiphilic compounds, which include compounds having both hydrophobic and hydrophilic regions. Indeed, molecules may contain water-loving (hydrophilic) moieties, such as the hydroxyl group in sterols and the carboxylic acid group in long chain fatty acids. This is true for many (biologically) active species for which embodiments of this invention provides compositions and methods for aqueous dispersion formation. In such cases the molecules may also be described as amphiphilic. The methods and compositions of this invention encompass entities that may be so described and that can be dispersed in hydrated lecithin vehicle bilayers; some embodiments are shown in the examples. In general, amphiphilic molecules are arranged in both portions of the bilayer, with their hydrophilic portions associated with the polar surface and the hydrophobic portions directed to the acyl chains of the phospholipids in the bilayer interior.

As used herein, dispersion refers to a disperse lecithin-based phase generally uniformly distributed in the bulk aqueous solution. Further, as used herein the dispersion is stable if it does not suffer from physical instability manifested by visible phase separation , such as when the vesicles aggregate and separate by precipitation or creaming (i.e. aggregates fall to the bottom or rise to the top of the mixture, respectively) or when the incorporated lipophilic materials separate from the vesicles and form visible aggregates. That is, in a stable dispersion of HLCV without an active ingredient, substantially all of the vesicles in the dispersion are distributed without visible clumping. For a stable composition including an active ingredient, in addition to the vesicle stability discussed above, the active ingredient remains properly associated with the vesicles. For example, if a lipophilic compound is incorporated in an HLCV dispersion, the hydrophobic regions of the lipophilic compound are associated with (in contact with) the non-polar regions of the phospholipid vesicle membrane. For a composition including lipophilic compounds having hydrophilic moieties incorporated in an HLCV dispersion, the hydrophilic regions of the lipophilic compounds are associated with the polar regions and the hydrophobic regions are associated with the non-polar regions of the phospholipid vesicle membrane. As such, a stable HLCV having an incorporated active ingredient also means that substantially all of the active ingredient present in the composition is incorporated into/associated with the vesicle membrane. The vesicle carrier having an active ingredient in its membrane is also referred to as a “loaded” carrier vesicle, and refers specifically to the active ingredient being incorporated in the phospholipid membrane of the vesicle to stabilize the compound from aggregation or degradation in the bulk aqueous solution. Accordingly, the active ingredient incorporated in the phospholipid membrane environment, and so dispersed in an aqueous medium, is stabilized in other environments (e.g., food, beverages, gastro-intestinal or digestive tract).

As used herein, conditioned water is defined as water that has less than 100 ppm hard ions, and in some embodiments less than 60 ppm hard ions. Hard ions cause hardness in water, and the free hard ions commonly found in water are calcium and magnesium ions. Conditioned water refers to water having a reduced level of free hard ions whether the water has a reduced level without treatment (i.e. “soft” water) or if treatment is required. Hardness is measured by an EDTA titration method such as that described in ASTM method D1126 “Standard Test Method for Hardness in Water.” The treatment to reduce hardness may include chelation. Naturally soft water, (i.e., water having hardness less than about 100 ppm of hard ions), is considered to be conditioned water for the purposes of this disclosure. In some embodiments, the water is essentially free of buffer ions and in other embodiments, the water has been purified by distillation, deionization, reverse osmosis or a similar technique such that the conductivity is less than 20 microSiemens per centimeter. Buffer ions are those that resist changes in their pH, for example phosphate, citrate, acetate and tris(hydroxymethyl)methylamino ions. Embodiments of the present invention recognize that the quality of the water has an effect on the final dispersion and stability of the lecithin vesicles. Compositions having unstable vesicles and/or vesicles (even if stable) having a broad or skewed size distribution can result in cloudy (turbid) dispersions. Accordingly, though the addition or presence of stabilizing agents is not necessary, in the absence of stabilizing agents, the likelihood of the HLCVs to cause whitening is inversely dependent on water purity—i.e., as water purity increases, the likelihood of whitening decreases.

### Hydrated Lecithin Carrier Vesicles (HLCVs)

Embodiments of the present invention are directed to hydrated lecithin carrier vesicles in which one or more active ingredients are incorporated. The HLCVs are prepared using lecithin having a low PC content and conditioned water (CW).

### Lecithin Having Low PC Content

The lecithin used to make the HLCVs has a low PC content. As used herein, lecithin having a low PC content means a lecithin ranging from non-deoiled (crude) lecithin to lecithin having a phosphatidylcholine content of approximately 80 w/w % or less. Indeed, in some embodiments, the lecithin has a PC content of less than, but not including 80 w/w %. For example, the HLCV dispersion can be prepared from known food-grade materials that are acceptable for consumption, including those listed as Generally Regarded As Safe (GRAS) by the US Food and Drug Administration. Accordingly, embodiments of the present invention have food-grade lecithin for which the phosphatidylcholine content is from about 20 to about 80 w/w %. In other embodiments, the lecithin has a phosphatidylcholine content from about 20 to about 70 w/w %. In other embodiments, the lecithin has a phosphatidylcholine content from about 20 to about 60 w/w %. In other embodiments, the lecithin has a phosphatidylcholine content from about 20 to about 50 w/w %. In other embodiments, the lecithin has a phosphatidylcholine content from about 20 to about 40 w/w %. In other embodiments, the lecithin has a phosphatidylcholine content from about 20 to about 30 w/w %. In some embodiments, lecithin that has not been de-oiled is used, for which the phosphatidylcholine content is from about 20 to about 25 w/w %.

### Active Ingredient

Non-limiting examples of lipophilic active ingredients include: olfactants, such as natural and synthetic fragrances and essential oils (which are described in more detail below); flavor compounds and taste modifiers, such as natural essences and essential oils, for example from apple, orange and lemon, (including combinations of such compounds with carrier oils); coloring agents, such as porphyrin based macrocycles; vitamins, such as vitamins A, D, E, and K and their pharmacologically active metabolites, salts and compounds, for example vitamin D, vitamin E acetate and vitamin A palmitate; phytochemicals, such as plant sterols and essential oils, for example beta-sitosterol, isoflavones, curcuminoids, and polyphenolic compounds; oil soluble acids and alcohols, such as lactylic acid and the essential fatty acids, for example linoleic and linolenic acids, eicosapentaenoic acid(20:5 n-3) and docosahexaenoic acid(22:6 n-3) and their natural sources, such as evening primrose oil, safflower oil and fish oil; drugs such as cyclosporin A, propofol, fat soluble protease inhibitor antiretroviral drugs, antibiotics and lipophilic members of other drug classes; carotenoids, such as beta-carotene and lycopene; steroidal hormones, such as estrogens, estradiols, and cortisones; flavonoids, such as resveratrol; proteins, enzymes, coenzymes and numerous other lipophilic biologically active compounds. It is obvious, however, to those of ordinary skill in the art, that the compounds are not limited to particular classes of lipophilic ingredients of foods, beverages, medicines and nutritional supplements.

As used herein, an essential oil is a concentrated, hydrophobic liquid containing volatile aroma compounds from plants. Essential oils do not necessarily as a group have specific chemical properties in common beyond conveying characteristic fragrances. They are well known for their use as olfactants and flavoring agents and find wide utility in traditional medicine. (Traditional medicine, as defined by the World Health organization, refers to the knowledge, skills and practices based on the theories, beliefs and experiences indigenous to different cultures, used in the maintenance of health and in the prevention, diagnosis, improvement or treatment of physical and mental illness).

### Alcohol

In some embodiments, the HLCV composition includes alcohol. In some embodiments, alcohol is added to the conditioned water for hydration of the lecithin to form the HLCVs. In other embodiment, if the active ingredient is not easily solubilized in the lecithin composition, the addition of alcohol can improve solubilization of the active ingredient in the homogeneous liquid mixture. According to embodiments of the present invention, the alcohol is a short chain alcohol. Examples of short chain alcohols include methanol, ethanol, isomers of propanol, and isomers of butanol. The amount of alcohol needed to facilitate solubilizing the active ingredient will vary depending on the type of alcohol and the particular active ingredient, and can be determined empirically by a person having ordinary skill in the art.

In some embodiments, the alcohol added to help dissolve the active ingredient in the lecithin to form a homogenous liquid mixture, is provided in a range from about 5 to about 50% alcohol by weight relative to the combined weight of the lecithin and the active ingredient. The addition of alcohol to the lecithin and active ingredient composition may be with or without heating, and with or without the addition of oil, as discussed herein.

In some embodiments, alcohol is added to the conditioned water for the hydration of lecithin. In some embodiments, the additional alcohol is an aliphatic short chain alcohol (e.g., methanol, ethanol, propanol, or butanol). The amount of alcohol can vary and will depend on the properties of the active ingredient (s). For example, for the hydration of lecithin, up to a total of 40% v/v of an alcohol can be present in the HLCV composition.

In some embodiments, the dispersion may be dried by standard industrial methods for example to a powder, granule or cake form.

### Stabilizing Agents

In other aspects of the invention, the HLCV compositions further include at least one stabilizing agent. Non-limiting examples of stabilizing agents include polysorbate (polyoxyethylene sorbitan monoesters), polyoxyethylene alkyl ethers (PAEs), and the like. The addition of a stabilizing agent is optional and will generally depend on the properties of the active ingredient(s) to be dispersed in the HLCVs. For some applications, addition of a polysorbate or PAE may increase stability. As such, the need for polysorbate or PAEs can be determined empirically by those of ordinary skill in the art.

As used herein, the term polysorbates includes the class of emulsifiers which are oily liquids derived from polyoxyethylene derivatized sorbitan (a derivative of sorbitol) monoesterified with fatty acids. The PAE class of molecules is suitable for use in applications not involving ingestion of the HLCVs. It is readily apparent to those of ordinary skill in the art that, for applications not involving ingestion, a suitable PAE may be substituted for polysorbate in the methods and compositions described herein that include and/or employ polysorbates.

The polysorbate-containing HLCVs of the present invention do not have a detectable bitter taste and are physically stable to dilution, pasteurization and storage in water, many juices and other beverages, as demonstrated by retention of clarity. The following polysorbates are non-limiting examples that can be used: polyoxyethylene(20) sorbitan monooleate, polyoxyethylene(20) sorbitan monolaurate, polyoxyethylene(20) monopalmitate, and monostearate. In some embodiments, polyoxyethylene(20) sorbitan monooleate (i.e., polysorbate 80), or polyoxyethylene(20) sorbitan monolaurate are used. An effective amount of polysorbate can be determined using known methods. In some embodiments, for example, polysorbate is used at a molar ratio of polysorbate to lecithin of between about 1:3 and about 1:20. In other embodiments, polysorbate is used at a molar ratio of between about 1:5 and 1:10. In other embodiments, polysorbate is used at a molar ratio of between about 1:7 and 1:9. For the purpose of determining the molar ratio, the molecular weight of the lecithin is to be assumed to be 800.

### Methods of Preparing HLCV

Embodiments of the present invention are directed to methods of preparing hydrated lecithin carrier vesicles (HLCVs). In some embodiments, HLCVs with at least one active ingredient loaded therein are prepared without the use of any organic solvents, alcohols or otherwise. However, in some embodiments, alcohols may be used to facilitate making the HLCVs although other/additional organic solvents are not required or used. In some aspects of the present invention, a method of forming an HLCV composition includes hydrating and processing lecithin having a low PC content in conditioned water followed by the addition of at least one active ingredient. In other aspects of the present invention, the active ingredient may be mixed with low PC content lecithin and an alcohol, together with minimal water, to form a homogenous liquid phase (without forming vesicles), followed by hydration and processing which forms dispersed vesicles. In other aspects, methods of forming HLCV compositions having an active ingredient dispersed therein include using lecithin having a high PC content (i.e., greater than 80 w/w % phosphatidylcholine) in conditioned water.

### Hydration of Lecithin

In some embodiments, lecithin having a low phosphatidylcholine content is hydrated upon exposure to conditioned water to form hydrated lecithin carrier vesicles dispersed in CW. In aspects of the invention, the lecithin is hydrated with enough conditioned water to effectively perform a processing step (e.g. homogenization, sonication, microfluidization, high shear mixing, etc.). Indeed, the HLCV dispersions contain, by weight, at least as much conditioned water as lecithin, (i.e. a ratio of CW to lecithin of at least 1:1), prior to any drying or further compounding steps. In some embodiments, for example, lecithin may be hydrated with 3 parts water to 1 part lecithin (by weight). In other embodiments, lecithin may be hydrated with up to 4 parts water to 1 part lecithin, or 5 parts water to 1 part lecithin, by weight. In other embodiments, lecithin may be hydrated with greater than 5 parts water to 1 part lecithin. In these embodiments, the relative amounts of CW and lecithin are relative to the lecithin alone.

In other embodiments, lecithin is hydrated in the presence of at least one active ingredient. In these embodiments, the ratio of lecithin to active ingredient is at least 1:1 and may, in general, be up to about 5:1 by weight. However, there is no particular upper limit to this ratio other than imposed by commercial or practical processing constraints obvious to those of ordinary skill in the art. In these embodiments, the conditioned water is provided at a ratio of CW to the sum of lecithin and active ingredient ranging from about 3:1 to about 5:1, by weight. This is necessary as the active ingredient can be provided up to about a 1:1 ratio with the lecithin. For example, when the total weight of lecithin and active ingredient is 200 to 250 mg (lecithin+active ingredient), the amount of CW could be 1 ml (1000 mg). It is apparent to those of ordinary skill in the art that the amount of conditioned water with respect to the lecithin or lecithin and active ingredient, is, in general, only limited by the desired concentration of HLCV composition and active ingredient with respect to any further production or manufacturing steps for the final desired application of the composition.

### Loading of Active Ingredient in HLCVs

In some embodiments, HLCV compositions are prepared, (i.e., lecithin is hydrated and then processed) to form dispersed vesicles prior to the addition of at least one active ingredient. These methods of forming HLCVs prior to loading the active ingredient(s) are most effective when the active ingredient is a liquid (e.g., is in liquid form at a temperatures up to the boiling point of the conditioned water or the boiling point of the conditioned water and alcohol mixture), so that the active ingredient is easily solubilized in the hydrated lecithin vesicle composition. These methods of adding the active ingredient after processing (i.e., to pre-formed vesicles) are particularly suitable for such ingredients as essential oils, lipophilic flavor compounds, and flavor compound mixtures having lipophilic components. This method is most effective for HLCVs that have been processed (e.g. by homogenization or high shear mixing) to form UVs prior to loading. Accordingly, if the active ingredient can be incorporated in the hydrated lecithin composition, then the active ingredient can be added to “pre-formed” lecithin vesicles. As discussed above, solubility of the active ingredients may require the addition of alcohol, heating, or any combination of these. It is apparent to those of ordinary skill in the art that an active ingredient having a low solubility, if added in a small amount would slowly be incorporated in the vesicle composition over time. It is also known to those of ordinary skill in the art that the extent of incorporation in the dispersed HLCVs is dependent on both the rate at which the active dissolves in the aqueous phase and time. Without the aid of alcohol, or heating, an active ingredient having a log Kow (i.e., an octanol:water partition coefficient) of less than about 4.5, will not likely be incorporated within a reasonable amount of time. However, active ingredients having a log Kow of 4.5 or greater could be incorporated more rapidly. Solubility of such active ingredients, including as enhanced by heat and/or alcohol, can be determined empirically by those of ordinary skill in the art.

A stabilizing agent can be added to the lecithin composition prior to or after processing. In some embodiments, a stabilizing agent is added to the HLCV composition after processing. In other embodiments, a mixture of lecithin and a stabilizing agent is hydrated in CW prior to processing.

Following addition of the active ingredient to the HLCVs, the mixture is processed by high shear mixing or homogenization to form a dispersed composition of HLCVs having an active ingredient incorporated therein.

### Transparency

A further aspect of the present invention is that the size distribution of the lecithin carrier dispersion can be manipulated such that the dispersion is essentially optically clear (i.e. transparent). For the purpose of describing the invention herein, the mean diameter of dispersion particles and structures in the submicron range (<1,000 nm) is defined as the volume weighted mean diameter, generally of a unimodal distribution of sizes. The volume weighted mean diameter of the vesicles can be determined by any known technique. For example, the volume weighted mean diameter is determined using electron microscopy or dynamic light scattering. Upon determining the mean vesicle size, the vesicles can be reduced in size using standard methods well known in the art, including without limitation: sonication, microfluidization and high pressure homogenization.

In some embodiments, a lecithin carrier vesicle having an active ingredient therein and prepared by a method of the present invention, remains clear (or, transparent) in dispersion. Clarity refers to transparency rather than translucency. This transparency is achieved by producing a dispersion wherein the mean diameter of the particles is about 120 nm or less, preferably less than 100 nm, and more preferably less than 80 nm. Additionally, the distribution of sizes includes few particles of larger diameters that cause cloudiness, which may manifest as whitening. For the purpose of describing this invention, the presence of such larger particles may be quantitated by the cloudiness or haziness, hereafter referred to as turbidity. Transparent dispersions are those with low turbidity. The quantitation of such turbidity may be performed, for example, using a nephelometer. Turbidity of dispersions may be expressed relative to standards of known turbidity. The turbidity caused by scattering of light by submicroscopic particles, even those with diameters significantly smaller than the wavelength of the light, is a complicated function of many variables including both the particle size and the wavelength. In general, the presence of larger particles, i.e. those that cause turbidity (whitening, cloudiness, haziness), is revealed by scattering at longer wavelengths. This scattering of light results in the observed turbidity (i.e., lack of clarity) of aqueous dispersions. A quantitative measure of relative turbidity is the relative absorbance at 800, 860 and/or 900 nm as measured using a conventional UV/visible spectrometer. Turbidity caused by instability of the dispersions is thus readily quantitated by spectrophotometric methods in the desired range.

While HLCV compositions with or without dispersed active ingredients may desirably be transparent or nearly so, transparency is not a requirement of any embodiment of the present invention. For example, if the composition is intended to be added to a food product, its clarity in solution is typically not relevant, and therefore it may not be necessary to reduce the vesicle size. However, if the composition is intended to be added to a transparent or semi-transparent drink, for example, the cloudiness or turbidity may desirably be adjusted to meet consumer expectations.

### Homogenous Liquid Mixture

In some embodiments, the active ingredient is added to the lecithin prior to hydration. In these embodiments, the active ingredient may be dissolved at room temperature in the lecithin based mixture, which may also include alcohol and may also include a minimal amount of conditioned water and/or an oil. In some embodiments, the weight amount of lecithin in the homogenous liquid mixture is greater than any other single component of the homogenous liquid mixture. That is, while the lecithin may not be more abundant than all other components combined, it is provided in an amount that is more than any other single component. In this way, lecithin may be used as a solvent for the homogenous liquid mixture. Some active ingredients may more readily solubilize with heating. In some embodiments, therefore, the active ingredient is mixed with lecithin, and may include alcohol, a minimal amount of alcohol, oil, and may be mixed at an elevated temperature. For example, the heating temperature is selected from a range of about 60° C. to about 80° C. The desired temperature may be determined by one of ordinary skill in the art with consideration of the properties of the active ingredient and the components of the composition. For example, the heating temperature should not exceed the boiling point of the composition which is dictated by the various components of the composition. As would be understood by those of ordinary skill in the art, the heating temperature should not exceed the boiling point of the component of the composition which has the lowest boiling point. For example, if alcohol is present in the lecithin composition, then the highest desired heating temperature should not exceed the boiling point of the alcohol (which in general will be the component with the lowest boiling point). In some embodiments, lecithin and at least one active ingredient are first dissolved in a homogenous liquid mixture prior to vesicle formation.

By way of example, plant phytosterols have a melting temperature above 100° C. (e.g. beta-sitosterol has Tₘₚ of 136 to 140° C.), and therefore, plant phytosterols are not effectively incorporated into the HLCV composition after the vesicles are formed. Accordingly, in some embodiments of the present invention, a composition having at least one active ingredient dispersed therein, is prepared by first dissolving the active ingredient together with lecithin to form a single phase homogenous liquid mixture. In some embodiments, alcohol is added to help solubilize the active ingredient in the lecithin. In some embodiments, up to about 50% alcohol (by weight with respect to lecithin) is added to the lecithin and active ingredient mixture. As discussed, in some embodiments, a minimal amount of conditioned water may be added to aid in the solubilization of the active ingredient in the lecithin and alcohol mixture. For example, no more than 10% by weight (w/w) conditioned water relative to the weight of lecithin may be added. It is apparent to those of ordinary skill in the art that an excess of water will prevent the formation of a single phase. To further promote the formation of a homogeneous liquid mixture with the lecithin, the active ingredient may first be dissolved in an oil (with heating if necessary).

In some embodiments, the active ingredient may first be dissolved in an oil in order to facilitate solubilization in the lecithin to form the homogenous liquid mixture. For example, an active ingredient may first be dissolved in a non-polar, hydrophobic carrier substance, such as a natural oil, and then mixed with the HLCV composition. Dissolution of the active ingredient in oil may also be combined with heating and/or the addition of alcohol. Examples of an oil include extracted triglyceride seed oils from plants, such as soy, corn, olive, sunflower, canola, olive. Oils also include animal oils such as fish or krill, as well as essential oils. Essential oils include, without limitation, oils of: citronella, clove leaf, eucalyptus, grapefruit, lemon, lime, mentha arvensis/mint, orange, oregano, peppermint, spearmint, star anise, tangerine, tea tree, thyme and wintergreen, and the embodiments include the use of the primary chemical components of these oils, such as thymol, carvacrol, limonene, menthol, carvone, methyl salicylate, cineole, citranal, pinene, and terpinen-4-ol.

The homogeneous liquid mixture is then hydrated with mixing in CW to form vesicles. In some embodiments, a short chain alcohol is added to the CW, i.e., if the short chain alcohol is not already present in a sufficient amount in the lecithin containing mixture. A sufficient amount of short chain alcohol is no less than that which provides a final lecithin hydrating solution concentration of at least about 5 v/v% alcohol, and no more than about 40 v/v%. In some embodiments, the final lecithin hydrating solution concentration of alcohol is from about 20% v/v to about 30% v/v. The lecithin hydration is performed at a temperature that maintains the homogeneous liquid mixture with lecithin in a fluid state.

After formation of the HLCVs by hydration, the HLCVs are processed by homogenization or high shear mixing to form a dispersion of the active ingredient incorporated in the HLCV composition.

HLCVs from Lecithin Having a High PC Content

In further aspects of the invention, a method of producing an HLCV composition uses lecithin having a high PC content (i.e. lecithin having a phosphatidylcholine content of more than 80% w/w). In this method, high PC content lecithin (or alternatively, substantially pure phosphatidylcholine) is mixed with an active ingredient following one of the methods disclosed herein for solubilization of active ingredients in low PC content lecithin. The methods and intermediate compositions described herein for adding an active ingredient after hydration and processing, or by forming a homogenous liquid mixture prior to hydration and processing, are also applicable to the preparation of HLCVs using high PC content lecithin. Such HLCVs are suitable for uses in pharmaceutical applications. Indeed, in these embodiments, the lecithin has a higher PC content such that it is acceptable for pharmaceutical use. For example, in these embodiments, the HCLVs have 90 w/w % or greater PC by weight for an inhaled or injectable product.

Other purified phospholipids may be added as required for the desired in vivo performance of the formulation. In some embodiments, a pharmaceutically acceptable formulation of propofol for parenteral use may be made by hydrating an alcoholic mixture of phosphatidylcholine and phosphatidylglycerol in conditioned water, followed by homogenization with a high pressure homogenizer, and then incubation with propofol.

Optionally, a pharmaceutically acceptable stabilizing agent, such as polysorbate, may be added during hydration or after processing. The organic solvent-free processing method disclosed herein for lecithin is also applicable for pharmaceutically acceptable phosphatidylcholine. The alternative method, based on a homogeneous liquid mixture of lecithin and other ingredients, may be used with those pharmaceutically active ingredients that are soluble in the phosphatidylcholine-alcohol mixtures corresponding to those described with respect to the low PC content lecithin embodiments, with the addition of a small amount of conditioned water (up to 10% w/w relative to phospholipids) as required to generate a homogeneous liquid mixture. As described herein, the active ingredient may first be dissolved, with heating if necessary, in a pharmaceutically acceptable oil, such as a triglyceride ester of fatty acids (wherein the fatty acids may be the same or mixed).

### Further Processing, Purification

Following formation of the HLCVs the composition can be further processed as desired. For example, to reduce the vesicle size, the dispersion can be subjected to high shear mixing or high-pressure homogenization. The energy required for size reduction is reduced by the presence of alcohol, compared to the corresponding size reduction process performed, if feasible, on the same components in the absence of alcohol. The lower energy processing is of commercial benefit resulting in lower process energy costs and the ability to use a wider range of processing equipment. For example, with alcohol present, a greater degree of size reduction can be achieved for a given energy input; in some cases this enables production of an optically clear presentation of HLCVs whereas such optical clarity may not be achievable in the absence of the alcohol.

The HLCV compositions as disclosed herein can be dried to a solid form, for example to a powder, flake or cake, by any standard industrial drying method, such as spray drying or freeze drying, and alternatively or subsequently incorporated into a paste or cream. Additional further processing steps may include: adjusting the pH of the composition, addition of preservatives or antimicrobial agents, or the addition of flavors to enhance the taste of the composition.

### Applications

The HLCV dispersions according to embodiments of the present invention are essentially free of non-dispersed active ingredients (i.e., once dispersed in the HLCV composition, the active ingredients remain substantially dispersed and do not precipitate out of the dispersion to any significant degree). In some embodiments, the lecithin vesicle compositions of the present invention are distinct from nanoemulsions and from dispersions of non-bilayer solid nanoparticles stabilized by a surface active agent (such as lecithin).

The compositions, intermediate solutions, and methods for production, of certain aspects of this invention provide for aqueous based dispersions of water-insoluble materials using relatively inexpensive food-grade lecithins, specifically lecithin having a PC content of less than about 80% by weight.

Though not limited to any applications, the HLCVs according to certain embodiments of the present invention may be used to make substantially clear aqueous dispersions of fat-soluble active ingredients that may be used in beverages or nutritional supplements, as the dispersions are physically stable to dilution, pasteurization and storage in water, juices and other beverages. The fat-soluble ingredients can include antioxidants (for example, vitamin E). The dispersion can be concentrated and dried to a powder and rehydrated as required by the desired application. The compositions and methods of the invention also disclose use of food grade materials, for example, those that have already been qualified in an application as Generally Regarded As Safe by the US Food and Drug Administration.

The HLCV compositions of this invention can be processed using conventional equipment that is widely employed in the food and beverage industry. The components of the hydrated lecithin carrier vehicles are relatively inexpensive. In addition, the loading of the food/beverage/nutritional supplement ingredient into the carrier can be elevated to levels that yield cost effective formulations for use in relevant consumer and other products.

The HLCV dispersions of the present invention effectively behave as true solutions and may be, or may be employed in the preparation of, products that are to be consumed orally or otherwise introduced into the oral cavity. A particular benefit of some of the compositions of this invention is that they provide aqueous dispersions that are essentially optically clear as used in final products, and maintain that clarity with storage, with addition to some juices and other beverages, and with exposure to high temperature, as during pasteurization. In some embodiments, the lecithin-based matrix also provides enhanced chemical stability of the water-insoluble materials, e.g., resistance to oxidation, and can inhibit undesirable odors and taste, and poor mouth feel of these materials. Certain lipids are themselves considered to be food or nutritional supplement ingredients of choice, for example phosphatidylcholine and, especially, lipids derived from marine organisms, such as hill. It is clear that the HLCVs, intermediates, and methods of preparation described herein, may optionally employ these lipids.

The following Examples are presented for illustrative purposes only, and do not limit the scope or content of the present application.

### EXAMPLES

### Example 1-1

### HLCV Dispersion of Fish Oil (Omega-3 Formulations)

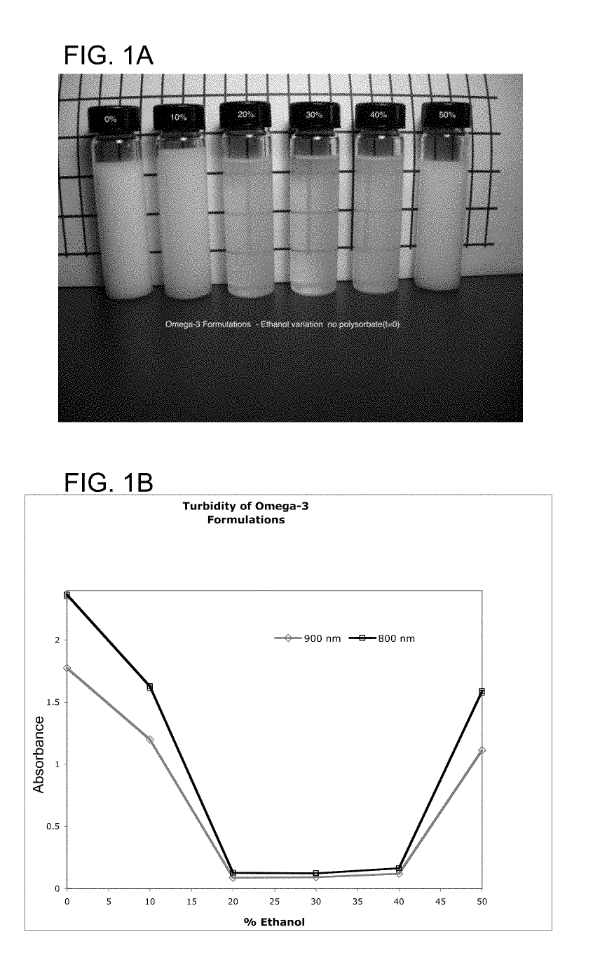

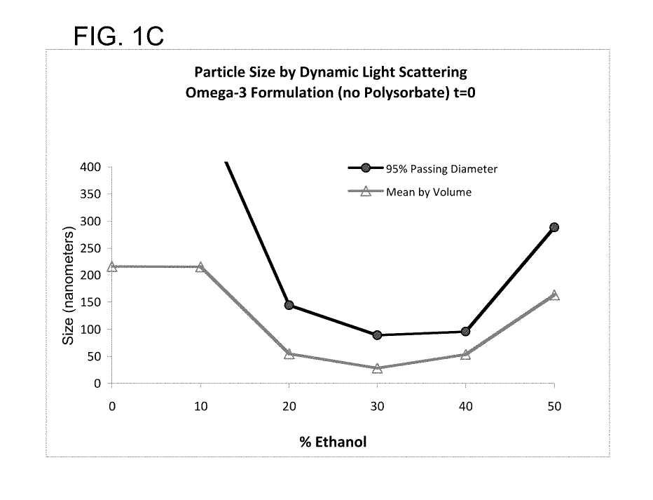

20 g fish oil (EPAX 1050), 30 g lecithin (Cargill Lecigran), 0.5 g Vitamin E, and 4 ml ethanol were combined in a bottle. The mixture was mixed with heating to 65C. for about 60 minutes until the solution was homogeneous in appearance. This mixture was added to 250 ml of purified water that had been heated to 65° C. and was mixed for 5 minutes. 50 ml samples of this hydrated fish oil lecithin dispersion were combined with suitable volumes of ethanol to produce dispersions containing approximately 0, 10, 20, 30, 40 and 50% (v/v) ethanol. For the purpose of determining the amount of ethanol to add, the small volume already present (from the homogeneous lecithin phase) was ignored. Each dispersion in this series was homogenized (Niro Soavi NS1001L homogenizer) at approximately 600 bar and 65° C. inlet temperature for 4 passes. Dispersions were then diluted in conditioned water to approximately 3 mg/ml fish oil. FIG. 1A shows a photograph of these dispersions. Turbidity of these dispersions was measured at 800 and 900 nm as shown in FIG. 1B. Particle size distribution measurements were obtained at ambient temperature using a Microtrac (USA) ‘Nanotrac 150’ dynamic light scattering instrument operating with a 780 nm solid state laser and performing controlled reference method (Doppler shift) analysis of reflected light intensity fluctuations. The volume-weighted mean diameter of the vesicles of the compositions are shown (FIG. 1C). As shown, the volume weighted distribution is plotted together with the 95% passing diameter (wherein 95% of the vesicles are smaller than this diameter). The volume weighted mean diameter is at a minimum at 30% ethanol (about 28 nm).

### Example 1-2

### The HLCV Omega-3 dispersions were made as in Example 1-1, except

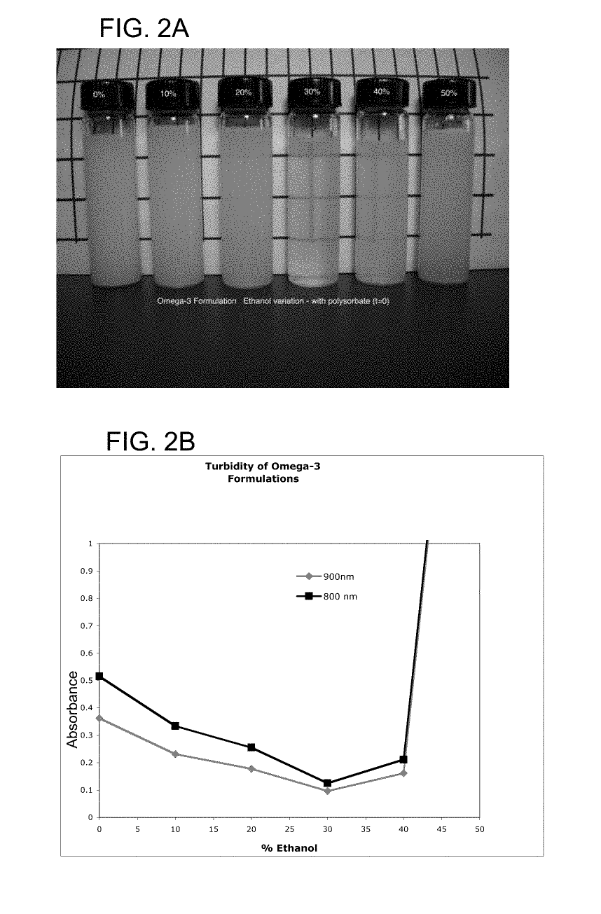

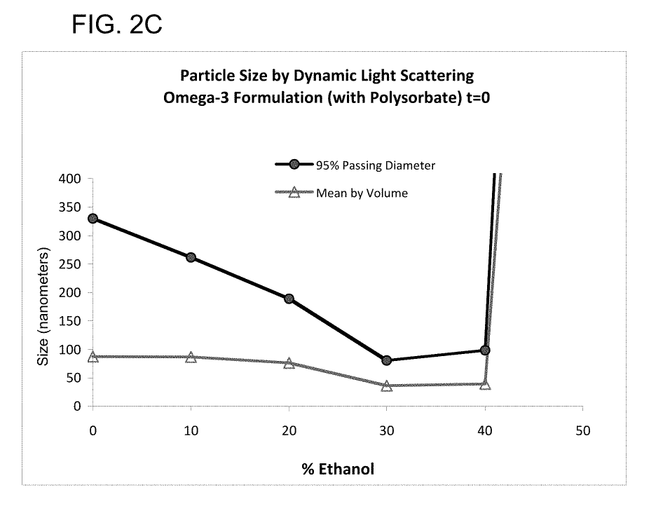

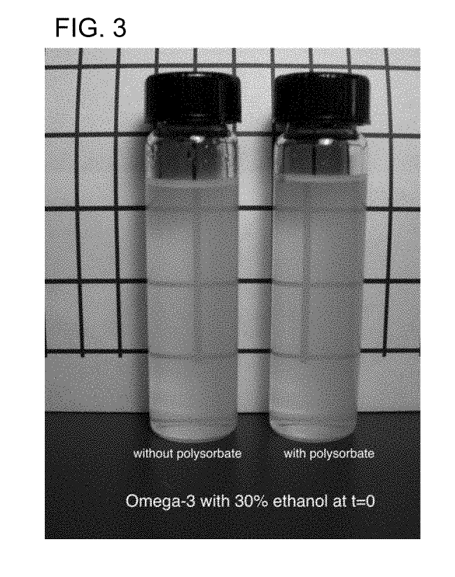

1.0 g of polysorbate 80 was added. A photograph of each of these Omega-3 formulations with polysorbate at 0, 10, 20, 30 40 and 50% ethanol is shown in FIG. 2A. Turbidity and particle size distribution data from dynamic light scattering for these formulations are shown in FIG. 2B and 2C, respectively. As shown, the smallest volume weighted mean diameter, at 30% ethanol, with polysorbate is about 35 nm. FIG. 3 shows a side by side photograph of these Omega-3 formulations at 30% ethanol with and without polysorbate.

### Example 2

### HLCV Dispersion of Essential Oils

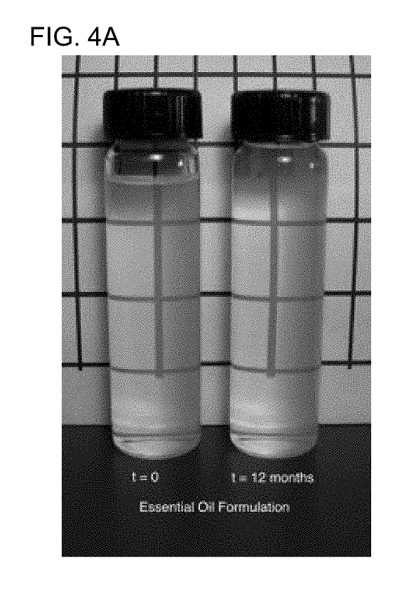

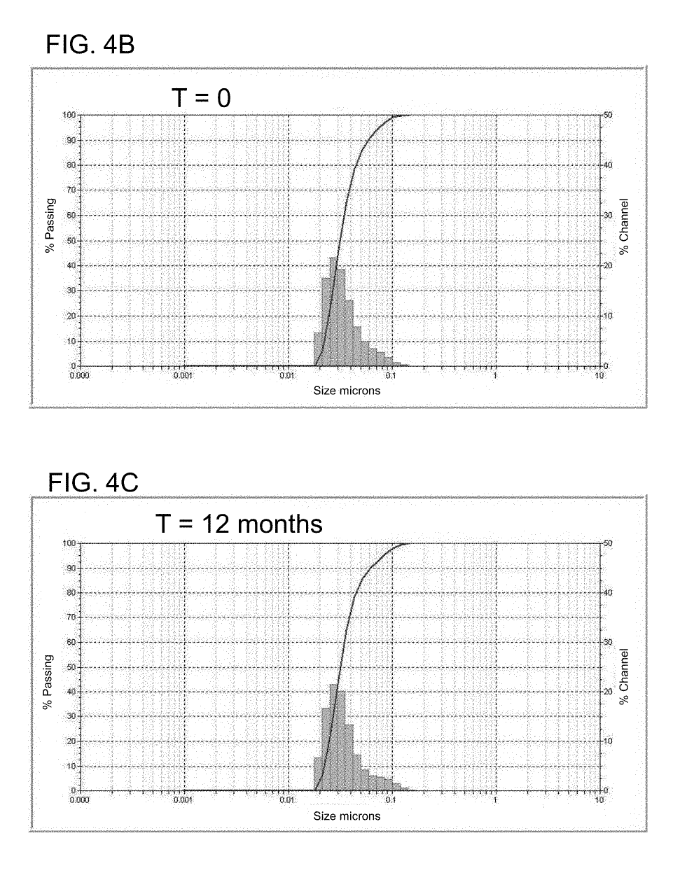

10 g of Lecithin (Lecigran, Cargill or Ultralec, ADM) was hydrated with mixing in 100 ml distilled water and then homogenized at room temperature with 6 passes at approximately 400 bar through a Niro Soavi NS1001L homogenizer to form HLCVs. Polysorbate 80 was added with mixing at a weight ratio of lecithin:polysorbate of 5:1. The dispersion was then diluted to 200 ml using distilled water. The essential oils: thymol (0.7 g), eucalyptol (1.0 g), methyl salicylate (0.65 g) and menthol (0.46 g), were added and mixed to produce an essentially transparent aqueous dispersion of these essential oils. The resulting dispersion was filtered through a 0.2 micron filter. The transparent dispersion was diluted and sweeteners added to produce an ethanol-free essential oil mouthwash. A photograph of two samples of the essential oil dispersion are shown in FIG. 4A, one freshly prepared (t=0) and one stored at room temperature for 12 months (t=12 months). Results of the Nanotrac particle size distribution analysis for these samples are presented in FIG. 4B and FIG. 4C. The volume-weighted mean diameter was determined to be 37 (+/−) nm in each case. For comparison, FIG. 4. shows the cumulants (the sigmoidal curves superimposed on the distribution histograms); and that the sizing data for the two samples are essentially indistinguishable.

### Example 3

### HLCV Dispersion of Lemon Oil

5 g Lecithin (Lecigran, Cargill or Ultralec, ADM) was hydrated with mixing in 100 ml conditioned water then homogenized at a pressure of approximately 400 bar at an inlet temperature of 65° C. with four passes through a Niro Soavi NS1001L homogenizer. Polysorbate 80 was added with mixing at a weight ratio of lecithin:polysorbate of 5:1. Lemon oil was added and mixed at a weight ratio of lecithin:lemon oil of 5:1 to produce an HLCV dispersion of lemon oil. The essentially transparent resulting solution can be filtered through a 0.2 micron filter.

### Example 4

### HLCV Dispersion of Lemon Oil in a Beverage

6 g of Lecithin (Lecigran, Cargill or Ultralec, ADM) was hydrated with mixing in 100 ml of distilled water and then homogenized at a pressure of approximately 400 bar at an inlet temperature of 65° C. with four passes through a Niro Soavi NS1001L homogenizer. 1.2 g of Polysorbate 80 was added with mixing at a weight ratio of lecithin:polysorbate of 5:1. Lemon oil was added and mixed at a weight ratio of lecithin:lemon oil of 5:1. After mixing, the dispersion was again processed with four passes under the same conditions through the same homogenizer. The resulting essentially transparent dispersion can be filtered through a 0.2 micron filter. The transparent dispersion was added to a solution of sucrose and citric acid to make a lemon flavor drink.

### Example 5

### HLCV Dispersion of Plant Sterols

10 g lecithin (Lecigran, Cargill or Ultralec, ADM) and 2 g plant sterols (Cardioaid, ADM or Corowise, Cargill) were dissolved in 1 ml of 90% ethanol at approximately 70° C. giving a weight ratio of lecithin:sterol of 5:1. Once all components were dissolved, the solution was added with mixing to distilled water followed by homogenization at an inlet temperature of 65° C. with six passes through in an APV Rannie 7.30.VH homogenizer at approximately 600 bar to produce plant sterol HLCVs.

### Example 6

### HLCV in Aqueous Beverage

The HLCV dispersion of Example 1-1 was combined with mixing at room temperature with polysorbate 80 at a weight ratio of 5:1 lecithin:polysorbate. The dispersion was then diluted in a sports drink to produce a clear (low turbidity) aqueous beverage containing fish oil.

### Example 7

### Marine Organism Lipids

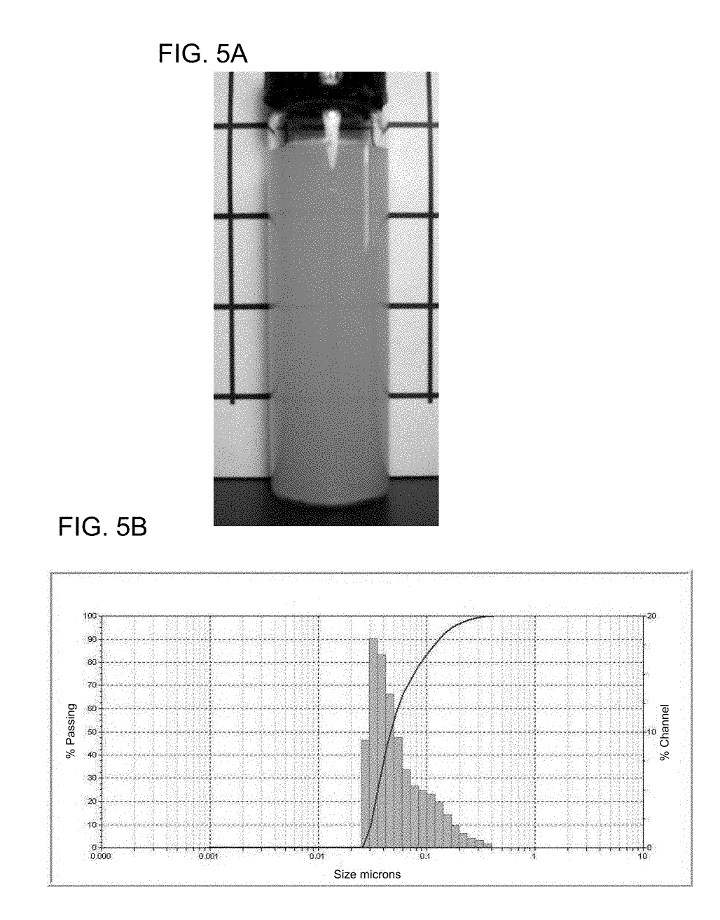

20 g hill oil (oil extracted from Euphasia superba), 30 g lecithin (Cargill Lecigran), 0.5 g Vitamin E, 1.0 g polysorbate 80 and 4 ml absolute ethanol were combined in a bottle. The mixture was mixed with heating to 65° C. for about 60 minutes until the solution was homogeneous in appearance. This mixture was added to 400 ml of purified water that had been heated to 65° C. and was mixed for 5 minutes. The dispersion was homogenized at approximately 600 bar and 65° C. inlet temperature for 6 passes through a Niro Soavi NS1001L homogenizer. The resulting dispersion was translucent and had a pink color characteristic of krill oil. A photograph of, and particle sizing data, for this hill oil HLCV composition are shown in FIGS. 5A and 5B, respectively. The volume weighted mean diameter was found to be 64 nm.

### Example 8

### Fish oil in Ethanol, Isopropyl Alcohol or Isobutyl Alcohol

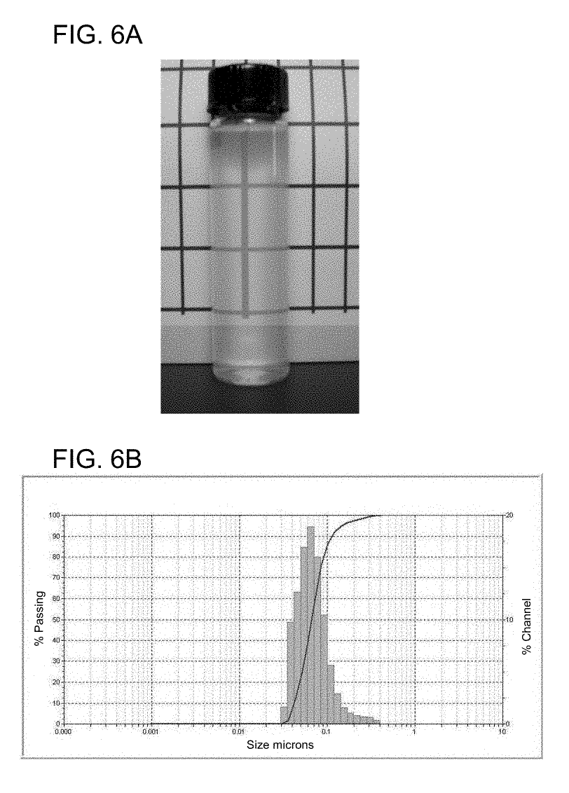

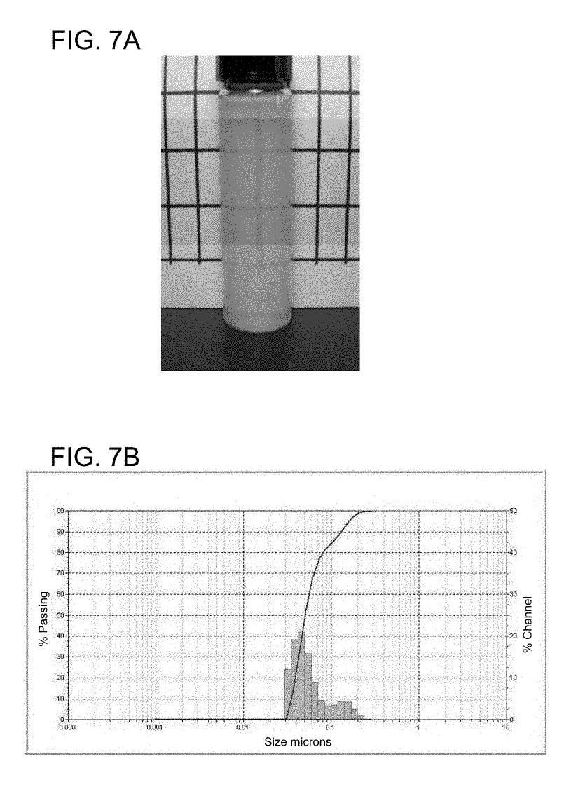

20 g fish oil (EPAX 1050), 30 g lecithin (Cargill Lecigran), 0.5 g Vitamin E, 1.0 g polysorbate 80 and 4 ml ethanol were combined in a bottle. In each case, the composition was mixed with heating to 65° C. for about 60 minutes until the solution was homogeneous and monophasic in appearance. These mixture was added to 250 ml of purified water that had been heated to 65° C. and was mixed for 5 minutes. 50 ml samples of the hydrated fish oil and lecithin dispersions were combined with additional purified water and alcohol, to produce hydrated dispersions in approximately 30%(v/v) ethanol, 30%(v/v) isopropyl alcohol, or 5% (v/v) iso-butyl alcohol in a total volume of 100 ml. Each dispersion was homogenized at approximately 600 bar and 65° C. inlet temperature for 4 passes through a Niro Soavi NS1001L homogenizer. Dispersions were diluted in purified water to approximately 3 mg/ml fish oil. A photograph of and particle sizing data for the iso-propyl alcohol fish oil HLCV composition are shown in FIGS. 6A-6B, respectively. A photograph of and particle sizing data for this iso-butyl alcohol fish oil HLCV composition are shown in FIGS. 7A-7B, respectively. FIG. 3 shows the appearance of the 30% ethanol sample.

### Example 9-1

### Essential oil: Carvacrol (without polysorbate)

10 g of lecithin (Cargill lecigran) was diluted in 200 ml of purified water heated to 65° C. The dispersion was mixed and hydrated for 10 minutes and then homogenized at approximately 550 bar and 65° C. inlet for 6 passes through a Niro Soavi NS1001L homogenizer. To this dispersion lg of carvacrol was added, the solution maintained at 65° C. and mixed for one hour and then allowed to cool to room temperature with mixing producing a dispersion of carvacrol in HLCVs

### Example 9-2

### Essential oil: Carvacrol (with polysorbate)

The HLCV composition of carvacrol was made as in Example 9-1, except after homogenization, 1 g of polysorbate 80 was added and the dispersion stirred for 10 minutes, followed by the addition of lg of carvacrol with mixing for one hour at 65° C. The dispersion was then allowed to cool to room temperature with mixing to produce a dispersion of carvacrol in HLCVs.

### Example 9-3

### Essential oil: Lecithin Control

The HLCV composition as in Example 9-1 was prepared without the addition of carvacrol.

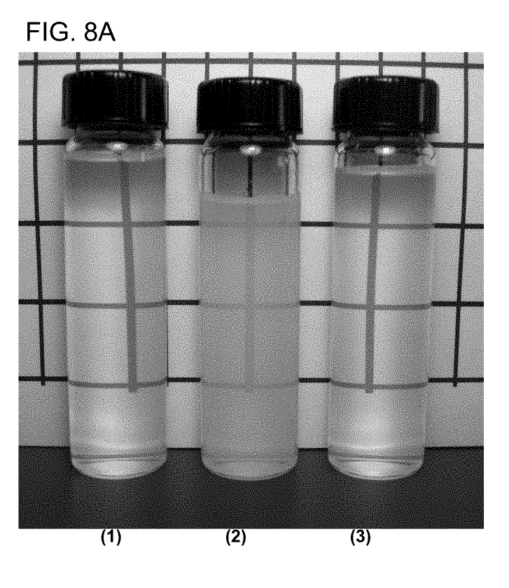

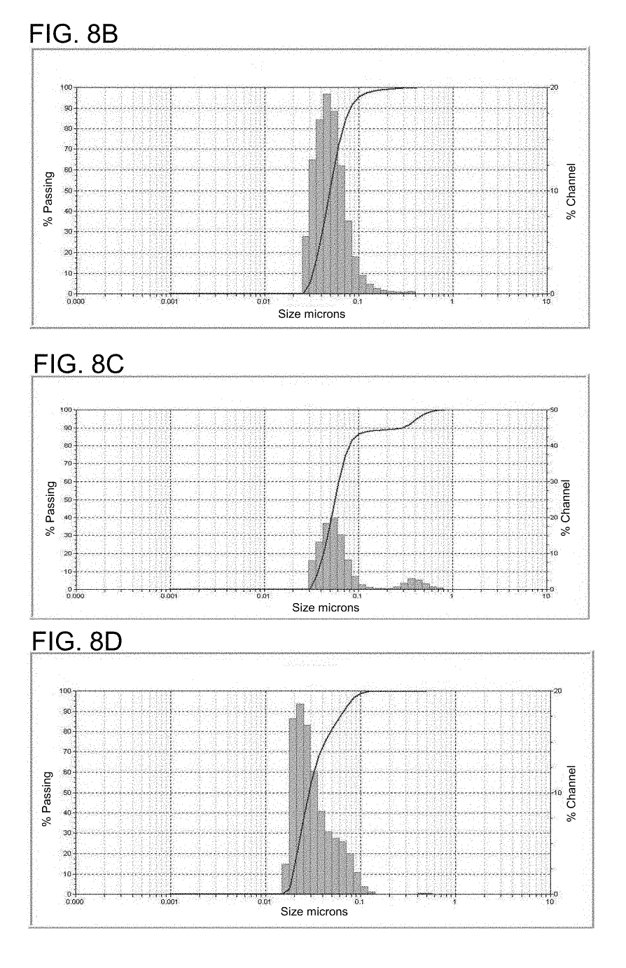

FIG. 8A is a photograph of the HLCV compositions from Examples 9-1, 9-2 (2) and 9-3 (3). The carvacrol sample prepared with polysorbate 80 (1) is very similar in appearance to the lecithin control (3). Although somewhat less transparent, the appearance of the carvacrol sample without polysorbate 80 (2) clearly demonstrates complete dispersion of the carvacrol in HLCV without the need for polysorbate 80. FIGS. 8B, 8C, and 8D show particle sizing data from Examples 9-1 (1), 9-2 (2), and 9-3 (3), respectively., for which the volume weighted mean diameters are 56 nm, 97 nm and 37 nm respectively. FIG. 8C suggests the presence of a skewed distribution with a few larger diameter particles present when polysorbate is not used—this corresponds well both to the reduced transparency seen in FIG. 8A and to the larger mean (by volume) diameter. It is, however, important to note that the submicron particle sizing by dynamic laser light scattering is suitable for demonstrating quantitatively the mean size but only qualitatively the similarities and differences in the distribution of particle sizes about the mean (i.e., the histogram plots reveal a difference but should not be relied upon to provide more information).

As discussed throughout and exemplified in the Examples and figures, HLCVs having a low PC content are loaded with active ingredients in the presence of conditioned water (and may also include alcohol and/or a stabilizing agent) to form a dispersed composition. The photographs of FIGS. 1A, 2A, 3, 4A, 5A, 6A, 7A and 8A show the disclosed examples of active ingredients loaded into HLCVs, as supported by the corresponding particle sizing data in FIGS. 1C, 2C, 4B, 4C, 5B, 6B, 7B, 8B, 8C, and 8D.

While the present invention has been illustrated and described with reference to certain exemplary embodiments, those of ordinary skill in the art will understand that various modifications and changes may be made to the described embodiments without departing from the spirit and scope of the present invention, as defined in the following claims.

1.-63. canceled

64. A composition, comprising:
a homogenous liquid mixture of:
a complex mixture of acetone-insoluble phosphatides, the complex mixture being derived from hydration of solvent-extract oils from an animal or plant source and comprising 80 w/w % or less phosphatidylcholine, the complex mixture being present in the homogenous liquid mixture in an amount greater than any other single component;
a lipophilic active ingredient; and
an alcohol.

65. The composition of claim 64, further comprising conditioned water.

66. The composition of claim 65, wherein the conditioned water has less than 100 ppm hard ions and a conductivity of less than 20 microSiemens per centimeter.

67. The composition of claim 65, wherein the conditioned water is present in an amount of about 10% or less by weight relative to the complex mixture.

68. The composition of claim 64, further comprising an oil.

69. The composition of claim 64, wherein the alcohol is present in an amount of about 5% to about 40% by volume.

70. The composition of claim 64, wherein the alcohol is an aliphatic alcohol having 6 carbons or fewer.

71. The composition of claim 64, wherein the alcohol is an aliphatic alcohol selected from the group consisting of methanol, ethanol, isomers of propanol, and isomers of butanol.

72. The composition of claim 64, wherein the lipophilic active ingredient is at least one lipophilic member selected from the group consisting of olfactants, natural essences, coloring agents, vitamins, vitamin salts, pharmacologically active vitamin metabolites, pharmacologically active vitamin metabolite salts, phytochemicals, oil-soluble acids, oil-soluble alcohols, essential fatty acids, primrose oil, safflower oil, lipids from marine organisms, cyclosporin A, propofol, fat-soluble protease inhibitors, antiretroviral compounds, antibiotics, carotenoids, steroidal hormones, flavonoids, proteins, enzymes, coenzymes, paints, inks, and agrochemicals.

73. The composition of claim 64, wherein the lipophilic active ingredient is selected from fish oil, krill oil, and/or an essential oil, the essential oil being a hydrophobic liquid having aroma compounds derived from plants.

74. The composition of claim 64, wherein the lipophilic active ingredient comprises Omega-3.

75. The composition of claim 64, wherein the complex mixture is alcohol-insoluble.

76. The composition of claim 64, wherein the complex mixture comprises 70 w/w % or less phosphatidylcholine.

77. The composition of claim 64, wherein the complex mixture comprises 60 w/w % or less phosphatidylcholine.

78. The composition of claim 64, wherein the complex mixture is present in an amount by weight greater than any other component of the homogenous liquid mixture.

79. The composition of claim 64, further comprising a stabilizing agent.

80. The composition of claim 79, wherein the stabilizing agent is a polysorbate or a polyoxxyethylene alkyl ether.

81. A method of forming a homogenous liquid composition, comprising:
mixing a complex mixture with a lipophilic active ingredient and an alcohol, the complex mixture being a complex mixture of acetone-insoluble phosphatides derived from hydration of solvent-extract oils from an animal or plant source and comprising 80 w/w % or less phosphatidylcholine.

82. The method of claim 81, further comprising mixing conditioned water with the mixing of the complex mixture with the lipophilic active ingredient and the alcohol.

83. The method of claim 81, wherein the alcohol is present in an amount of about 5% to about 40% by volume.
